(19) United States
(12) Patent Application Publication
(10) Pub. No.: US 2025/0092366 A1
(43) Pub. Date: Mar. 20, 2025

(54) Proteolytic Enzymes for Cell Culture Media

(71) Applicant: EFM Biotech Inc., San Antonio, TX (US)

(72) Inventors: Rodolfo Faudoa, San Antonio, TX (US); Elizabeth Faudoa, San Antonio, TX (US)

(21) Appl. No.: 18/580,802

(22) PCT Filed: Jul. 18, 2022
(86) PCT No.: PCT/US2022/037449

§ 371 Date: Jan. 19, 2024

Related U.S. Application Data
(60) Provisional application No. 63/223,950, filed on Jul. 20, 2021.

(51) Int. Cl. C12N 5/077

(52) U.S. Cl. CPC C12N 5/0658; C12N 2500/25; C12N 2500/32; C12N 2500/34; C12N 2500/38; C12N 2501/115; C12N 2501/195; C12N 2501/33; C12N 2501/734

(57) ABSTRACT

A composition is disclosed. The composition includes a cell culture medium; a proteinase; and a growth promoting factor. The doubling time of cells in the medium is the same or lower as compared to the doubling time of the cells in an otherwise identical composition which lacks the proteinase and has a higher concentration of the growth promoting factor. A method is also disclosed. The method includes growing the cells in a composition. The composition includes a cell culture medium; a proteinase; and a growth promoting factor. The doubling time of cells in the medium is the same or lower as compared to the doubling time of the cells in an otherwise identical composition which lacks the proteinase and has a higher concentration of the growth promoting factor.

### CROSS-REFERENCE TO RELATED APPLICATIONS

This application is a national phase entry under 35 U.S.C. § 371 of International Patent Application PCT/US2022/037449, filed Jul. 18, 2022, designating the United States of America and published in English as International Patent Publication WO 2023/003804 on Jan. 26, 2023, claims priority to U.S. Provisional Patent Application 63/223,950 filed on Jul. 20, 2021, and titled Novel Defined Media Serum-Free Animal Component Formulations and Methods for Stem and Somatic Cells and for Efficiently Optimizing In-Vitro and In-Vivo Cell Systems, the entirety of which is herein incorporated by reference.

### TECHNICAL FIELD

Media for cell culture and propagation.

### BACKGROUND

Four decades ago, the emergence of the biotechnology industry sparked the development of methods for large-scale cell culture, as companies raced to produce therapeutic quantities of the first recombinant proteins. Continuous refinements to cell culture techniques, instrumentation, and quality control measures ultimately have made large-scale cell culture more a science than an art. Cell culture is vital not only to the development of new pharmaceuticals, but to the testing of existing therapeutics, the advancement of tissue engineering and transplant, and the rigorous understanding of biologic and physiologic systems, including cancer.

Current cell culture technologies are not physiological relevant because they usually overload the cell in order to achieve certain degree of cell proliferation and/or to keep the cells undifferentiated. However, this involves suppressing and interfering with the normal autocrine, paracrine, endocrine, and intercellular activities.

The anabolic role of intracellular metabolites and their derivatives in cell proliferation and differentiation has been well described. Cells in culture synthesize macromolecules including nucleotides, lipids, and proteins de novo; however, they are reliant on an exogenous source of circulating nutrients. As extrinsic factors in the microenvironment impact proliferation, phenotype, and gene transduction, the overall outcome of the ex-vivo culture process is highly dependent on the properties of the medium.

There are a number of growth limitations for normal cells grown in culture. To circumvent these limitations, researchers and biotechnology manufacturers often employ immortalized cell lines for the development, testing and manufacturing. There are many benefits of cell lines such as cost, reproducibility, and longevity in culture. Primary cell cultures are cultures of cells that are freshly isolated from intact tissues. These cells are often good source of cell culture material and are well suited as host cells. However, primary cells are not efficient and do not replicate consistently and exhibit a limited life span in culture, eventually under-going senescence. At senescence the cells cease to divide and die out in a matter of time. The ability of cells to divide over time in culture is dependent on several parameters including the species of origin of the cell and the age of the tissue when it was placed in culture. Cells that undergo senescence cannot be maintained in culture for long periods of time and therefore are not useful reproducible hosts for the growth of commercial cells stocks. Some primary cells escape senescence and acquire the ability to become immortal. Rodent cells appear to undergo spontaneous immortalization quite easily but normal human and avian cells are rarely shown to be capable of spontaneous immortalization. Cells can be induced to undergo immortalization following exposure to agents known to induce cell immortalization.

The identification of suitable cell lines for growth and to replace primary cells is also favored in view of U.S. Government Principles for the Utilization and Care of Vertebrate Animals in Testing, Research, and Training and the Animal Welfare Act (7 U.S.C. § 2131) stating, in part, that in all cases, methods such as in vitro biological systems should be considered in lieu of in vivo animal model systems.

There is a need for cells that are immortal and support cell growth for manufacturing of animal-free products, including but not limited to meat and leather. This invention includes the identification of spontaneously and induced immortalized cell lines. In particular, relates to immortalized cell lines derived from primary chicken fibroblasts, primary chicken myoblasts, porcine skin keratinocytes, porcine skin fibroblasts, bovine adipocytes and bovine fibroblasts.

This invention relates to cultures of primary cells and to immortalized subclones of the immortalized cell lines under animal-free, serum-free cell culture media that has been described herein, and that support growth and cell proliferation.

### SUMMARY

In a first aspect, the disclosure provides a composition. The composition includes a cell culture medium; a proteinase; and a growth promoting factor. The doubling time of cells in the medium is the same or lower as compared to the doubling time of the cells in an otherwise identical composition which lacks the proteinase and has a higher concentration of the growth promoting factor.

In a second aspect, the disclosure provides a method for growing cells. The method includes growing the cells in a composition. The composition includes a cell culture medium; a proteinase; and a growth promoting factor. The doubling time of cells in the medium is the same or lower as compared to the doubling time of the cells in an otherwise identical composition which lacks the proteinase and has a higher concentration of the growth promoting factor.

Further aspects and embodiments are provided in the foregoing drawings, detailed description and claims.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a graph comparing cells grown in serum-free media to cells grown in the same media but containing a proteinase.

FIG. 2 is a micrograph of cells grown in a serum-free media lacking a proteinase.

FIG. 3 is a micrograph of cells grown in serum-free media containing a proteinase.

FIG. 4 is a graph comparing cells grown in media containing 20% fetal bovine serum to cells grown in serum-free media and containing a proteinase.

FIG. 5 is a micrograph of cells grown in 20% fetal bovine serum.

FIG. 6 is a micrograph of cells grown in media containing a proteinase and 1 ng/mL of FGF-2 and 0.0025 ng/mL of TGFβ3.

FIG. 7 is a graph comparing number of cells obtained after a defined time (in millions of cells per ml) when grown in suspension in serum-free media containing standard concentration of growth factors to cells in serum free media with 50% less growth factors and a proteinase.

FIG. 8 is a graph comparing cells grown in serum-free media containing standard concentration of growth factors to cells in the same media with the addition of a proteinase.

### DETAILED DESCRIPTION

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

### Definitions

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to intro-duce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

This invention introduces and applies the physiological aspects of proteolytic enzymes to cell culture media formulations and their applications in the biotechnology industry, life sciences, and technology in general. The present invention incorporates the use of any type of proteinases that relates to improving a cell culture medium for culture, propagation, and efficiently growing of cells, and decreasing the amount of growth factors used in the culture, maintenance, growth, propagation and/or differentiation of any type of cells and from any type of species.

Proteolytic enzymes (also termed peptidases, proteases and proteinases) are recognized as an essential and ubiquitous for the regulation of a myriad of physiological processes. They are found in all living organisms, from plants, viruses to animals and humans. Although it has been surmised that proteases are degrading enzymes that recycled amino acids, most of the proteases in all organisms, especially complex metazoans such as ourselves, do not spend their time degrading proteins. Mostly, they create very limited cuts in target proteins as essential components of signaling pathways and networks. Proteases are extensively applied enzymes in several sectors of industry and biotechnology, furthermore, numerous research applications require the use of them, including the production of Klenow fragments, peptide synthesis, digestion of unwanted proteins during nucleic acid purification, cell culture tissue dissociation, preparation of recombinant antibody fragments for research, diagnostics and therapy, exploration of the structure-function relationships by structural studies, removal of affinity tags from fusion proteins in recombinant protein techniques, peptide sequencing, and proteolytic digestion of proteins in proteomics. However, proteinases have not been applied and/or formulated as a media component in any type of cell culture media formulation.

Current cell culture technologies are not physiological relevant because they usually overload the cell in order to achieve certain degree of cell proliferation and/or to keep the cell undifferentiated. However, this involves suppressing and interfering with the normal autocrine, paracrine, endocrine, and intercellular activities.

The anabolic role of intracellular metabolites and their derivatives in cell proliferation and differentiation has been well described. Cells in culture synthesize macromolecules including nucleotides, lipids, and proteins de novo; however, they are reliant on an exogenous source of circulating nutrients. As extrinsic factors in the microenvironment impact proliferation, phenotype, and gene transduction, the overall outcome of the ex-vivo culture process is highly dependent on the properties of the medium.

Prior to the invention described herein, it was not known how proteinases as a media component, alone or in combination with other components, affects various cell functions such as viability, proliferation, and differentiation in cell culture.

An abundance of literature over the past several years indicates a growing interest in the role of proteinases in normal physiology. Proteinases were originally defined by their ability to degrade the extracellular matrix, but it is now well documented that their substrates extend far beyond matrix components. Recent reviews discuss the structure and function of the MMP family members, as well as the promoter sequences that control gene expression. In addition, increasing data implicate MMPs as “good guys”, protective agents in some cancers and in helping to resolve acute pathologic conditions. Despite the intricate and complicated roles of MMPs in physiology and pathology, the goal of designing therapeutics that can selectively target MMPs remains a major focus. Developing MMP inhibitors with targeted specificity will be difficult; success will depend on understanding the role of these enzymes in homeostasis and on the careful delineation of mechanisms by which this family of enzymes mediates disease pathology.

The matrix metalloproteinases (MMPs) constitute a multigene family of over 25 secreted and cell surface enzymes that process numerous pericellular substrates. Their targets include other proteinases, proteinase inhibitors, clotting factors, chemotactic molecules, latent growth factors, growth factor-binding proteins, cell surface receptors, cell-cell adhesion molecules, and virtually all structural extracellular matrix degrading enzymes. Thus, MMPs are able to regulate many biologic processes and are closely regulated themselves. Recent reviews explain how MMPs work, how they are controlled, and how they influence biologic behavior. These advances shed light on how the structure and function of the MMPs are related and on how their transcription, secretion, activation, inhibition, localization, and clearance are controlled. MMPs participate in numerous normal processes, and there are new insights into the key substrates and mechanisms responsible for regulating some of these processes in vivo. The knowledge in the field of MMP biology is rapidly expanding, yet we still do not fully understand how these enzymes regulate most processes of development, and homeostasis.

Proteinases are required for numerous developmental processes. The ability to degrade extracellular proteins is essential for any individual cell to interact properly with its immediate surroundings and for multicellular organisms to develop and function normally. This was obvious long before it was first shown that diffusible enzymes produced by fragments of involuting tadpole tail could degrade gels made of native fibrillar collagen. Since then, a family of related enzymes has been identified in species from hydra to humans and collectively called matrix metalloproteinases (MMPs) because of their dependence on metal ions for catalytic activity, their potent ability to degrade structural proteins of the extracellular matrix (ECM), and specific evolutionary sequence considerations that distinguish them from other closely related metalloproteinases. In addition to their ECM substrates, MMPs also cleave cell surface molecules and other pericellular non-matrix proteins, thereby regulating cell behavior in several ways.

Matrix metalloproteinases (MMPs) are members of the large metzincin superfamily. In the classical view, MMPs are collectively capable of degrading all components of the extracellular matrix (ECM) and basement membrane, restricting their functions to tissue remodeling and maintenance. However recent substrate identification studies reveal that MMPs regulate the release or activation of chemokines, cytokines, growth factors, antibiotic peptides, and other bioactive molecules thus participating in physiological processes such as innate and adaptive immunity, inflammation, angiogenesis, bone remodeling, neurite growth, and wound healing.

High sequence similarity to MMP catalytic domains is found in almost all kingdoms of life. At least 25 different vertebrate MMPs have been characterized up to now and 24 different MMPs are found in humans, including the two identical forms for MMP-23, encoded by two distinct genes, i.e. MMP23A and MMP23B. The diversity of the current mammalian MMP gene families is derived particularly from an extensive gene tandem duplication and exon shuffling during evolution in the tetrapod lineages. Taking this into account, some of the actual MMP members are most likely derivatives from a single gene resulting in a MMP gene cluster, whose organization is preserved from amphibians to mammals.

MMPs has been shown to be essential in cell biological processes and many fundamental physiological events involving tissue remodeling, such as angiogenesis, bone development, wound healing, and mammary involution.

The regulation of MMP starts early on with their secretion. The extracellular environment and the localization of MMPs in the pericellular space generally have a strong impact on their inactive pro-form activation and proteolytic efficiency and specificity.

Secreted MMPs are often associated to the cell membrane, which focuses their activity to specific substrates in the pericellular space. Examples for cell surface substrate recruitment are binding of MMP-1 to α2β1 integrin, which depends on interaction of α2 integrin with both linker plus hemopexin-like domain of MMP-1.

Cells use surface receptors, like integrins to inform themselves what protein in the cell periphery has been encountered and consequently, which type of enzyme is needed and where it has to be released. This mechanism has been confirmed in vivo with MMP-1 in human cutaneous wounds, where this enzyme was induced in basal keratinocytes just at the moment when the cells were detached from the basement membrane and contacted type I collagen in the underlying dermis. Furthermore, it has been shown that this mechanism depends on the interaction of integrin α2β1 with type I collagen, which also triggers secretion of the enzyme to the points of cell-matrix contact.

MMPs are initially synthesized as inactive pro-forms (zymogens) with a pro-domain which has to be removed for activation. The pro-domain harbors a conserved “cysteine switch” sequence motif in close proximity to the border zone of the catalytic domain, whose free cysteine residue interacts with the catalytic zinc ion to maintain enzyme latency and prevent binding and cleavage of the substrate. The activation of the MMP zymogen depends on a conformational change in the pro-domain which pulls out the cysteine residue and enables water to interact with the zinc ion in the active site (3). Although MMP gene expression is primarily regulated at the transcriptional level, post-transcriptional control of mRNA stability by cytokines, nitric oxide or micro-RNA (miRNA) has been recently described as a significant contributing mechanism. Despite the low expression of most MMPs under quiescent conditions, their transcription is tightly regulated. No single cytokine, chemokine, oncogene or growth factor has been identified that is exclusively responsible for the overexpression of MMPs, although tumor necrosis factor (TNF)-α and interleukin (IL)-1 are often implicated. The signal-transduction pathways that modulate MMP promoter activities are also diverse. Several of the MMP promoters share several cis-elements in their promoter regions, consistent with observations that some MMPs are co-regulated by several cytokines and growth factors, like epidermal growth factor, keratinocyte growth factor, vascular endothelial growth factor (VEGF), platelet-derived growth factor, TNF-α, and transforming growth factor (TGF)-β, and may also be co-repressed by glucocorticoid hormones and retinoids.

MMP processing of ECM components can yield bioactive fragments. Bioactive cleavage products are also produced from ECM protein, such as perlecan, laminin or fibronectin. In addition to “shaping” bioactive cleavage products, ECM degradation releases non-covalently bound growth factors and cytokines and thereby increases their bioavailability. Examples include release of VEGF and TGF-β. Similarly, MMPs contribute to cytokine and growth factor bioavailability by proteolysis of soluble binding proteins, such as insulin-like growth factor-binding proteins and pleiotrophin, a VEGF masking protein. Important functions of shedding include release of bioactive protein domains and receptor processing, hence altering cellular responsiveness to growth factors and cytokines. MMPs are of outstanding importance in the site-specific cleavage of growth factors and cytokines. Through processing of these signaling molecules, MMPs interfere with cellular communication. The list of signaling-related MMP cleavage events is constantly growing and cytokine processing has now been recognized as pivotal MMP function in vivo. Further examples include processing stromal cell-derived factor 1, IL-8, IL-10, connective tissue growth factor and TNF.

In vivo as well as in vitro data have demonstrated the importance of the MMPs that contribute either directly or indirectly to the process of wound healing and neovascularization. Upon injury most, if not all, MMPs are induced and expressed in almost any involved cell types, including mesenchymal, epithelial and immune cells. The induction of MMP-1 expression by keratinocytes at the wound edge, for example, is mediated by the binding of α2β1 integrin to the dermal collagen type I. This high affinity attachment would tether keratinocytes to the dermis, rendering them unable to migrate. The increase of active MMP-1 leads to the subsequent degradation of dermal collagen type I. Collagen I degradation lowers the affinity of the integrin-collagen binding, thereby allowing the keratinocytes to migrate, and reduces the expression of MMP-1. This regulatory mechanism demonstrates that cells do not need MMP activity simply to remove matrix barriers.

Another recently identified role of MMP activity in wound healing is the recruitment of immune cells since neutrophil recruitment requires the presence and activity of MMP-7 and MMP-8.

A vital process in wound healing is neoangiogenesis, which involves migration and proliferation of endothelial cells, and is stimulated by fibrin and fibronectin derived from wound granulation tissue. MMPs induce the release of ECM bound pro-angiogenic factors, including the release of VEGF and TNF-α by MMP-2 and -9.

More work has to be invested in order to uncover the complex MMP interactions in a context dependent manner, to advance our understanding of the role of MMPs in the regulation of tissue homeostasis.

In summary, MMPs are multifunctional proteases that: 1) proteolyze ECM components with subsequent release of bioactive fragments and proteins; 2) participate in membrane shedding; 3) play an important role in chemokine processing; and 4) alter the activity status of other proteases.

Proteinases as part of media formulations.

The traditional opinion of proteinases being destructive aggressors that break down proteins for the purposes of elimination is evolving. Proteinases can, in fact, act as exquisitely sensitive switches in many biological processes. They activate growth factors and cell-surface receptors in many ways, which launch a host of important signal cascade pathways inside the cell.

The members of matrix metalloproteinase (MMP) family, share a catalytic domain coordinated by zinc, catalyzing the degradation of protein components of extracellular matrix (ECM), in their immediate environment, as well as activating latent growth factors, cell surface receptors, and adhesion molecules. Cell-ECM interactions trigger cellular signaling that promote cell proliferation, differentiation, survival, and migration, essential for normal cellular homeostasis.

Cells use a variety of surface receptors to sense the presence and location of specific molecules in the extracellular space. For example, integrin-ligand in the extracellular space, and in turn, these contacts activate signaling pathways involved in differentiation, proliferation, and gene expression, among other processes. During migration, cells need to proteolyze, to some extent, nearby extracellular matrix degrading enzymes, and hence, cell-matrix contacts instruct cells that proteinases are needed and should be released. An example of cell-matrix-mediated spatial regulation of proteolysis is seen with collagenase-1 (MMP-1), a matrix metalloproteinase, in human cutaneous wounds. In response to injury, collagenase-1 is induced in basal epidermal cells (keratinocytes) as the cells move off of the basement membrane and contact type I collagen in the underlying dermis, and this inductive response is specifically controlled by the collagen-binding integrin a2b1. It has been demonstrated that catalytic activity of collagenase-1 is required and sufficient for keratinocyte migration. For example, keratinocytes plated on mutant, collagenase-resistant type I collagen do not migrate, even in the presence of fibronectin and vitronectin; yet they express MMP-1 at levels equivalent to those released by cells on wild-type collagen. Keratinocyte migration is also completely inhibited by anti-collagenase-1 antibodies, which block the catalytic activity of the enzyme, and by anti-a2b1 blocking antibodies. It is becoming increasingly clear that extracellular proteolysis is a cell-regulated process.

After all, cells do not release proteases indiscriminately, especially enzymes. Rather they rely on precise interactions to accurately degrade, cleave, or process specific substrates.

Cell Culture more a science than an art.

The invention encompasses methods and compositions for the use of proteinases for cell culture media and for therapeutic and other purposes. In some embodiments, the proteinase is collagenase, such as matrix metalloprotease-1 (MMP-1) (also referred to herein as “collagenase-1” or “interstitial collagenase”). In cell culture embodiments, virtually any type of cell that may be cultured can be advantageously cultured using the methods and compositions of the invention; thus, the invention is useful in, e.g., primary cell culture and cell line culture. The methods of the invention allow long-term culture of cells with low or no contamination by fibroblasts.

Any desired cell type may be cultured in vitro in the presence of one of the culture media of the present invention. Non-exclusive examples of cell types that may be cultured include stem cells, progenitor cells, mesenchymal cells, epithelial cells, such as keratinocytes, cartilaginous cells, osseous cells, muscular cells, gland cells, fat cells, pericytes, satellite cells and dermal cells.

Forms of proteinases that may be used in the invention include, but are not limited to, collagenase isolated from cells, e.g., from bacterial cells, and synthetic collagenase, e.g., recombinant collagenase. As an illustrative example, the invention is often described in terms of the use of MMP-1, however, it is understood that any suitable proteinase may be used in the methods and compositions of the invention. MMP-1 that may be used in the compositions and methods of the invention include the entire native polypeptide (either in its final form or as a preprotein), as well as analogs, fragments, and modified forms of MMP-1, which are included in the term “MMP-1” as used herein. The MMP-1 may be from any source, including, but not limited to, bacterial, animal, mammalian, or human, and may be of natural origin synthetic, or recombinant.

The methods and compositions of the invention utilize a proteinase from plants. In cell culture aspects of the invention, the proteinase is a proteinase that is not produced by cells in the medium. In some embodiments, the proteinase is a partially or highly purified collagenase, e.g. a bacterial proteinase such as a collagenase isolated from Clostridium histolyticum. In some embodiments, the proteinases useful in the invention are matrix metalloproteases (MMPs, also called metalloproteinases). Exemplary MMPs useful in methods and compositions of the invention include MMP-1, MMP-2, MMP-8, MMP-9, and MMP-13, In some embodiments, the invention utilizes MMP-1. In addition, functionally active fragments, variants, and analogs of a collagenase, such as MMP-1, are also included within the term “collagenase,” or, in some embodiments, “MMP” or “MMP-1,” as used herein. An agent that induces the cell to increase its production of proteinase, e.g., MMP-1 (herein, a “proteinase-inducing agent” or an “MMP-1-inducing agent”) may also be used in some embodiments.

For convenience, the invention will sometimes be described with reference to MMP-1 as an exemplary proteinase, however, it is understood that any suitable proteinase may be used in the compositions and methods of the invention. MMP-1 is also known as matrix metalloproteinase-1, collagenase-1 and interstitial collagenase. MMP-1 from any source, natural or synthetic, may be used, and the MMP-1 may be the proenzyme or the active enzyme.

Matrix metalloproteinases (MMPs) are a large family of zinc proteinases that are secreted by both resident and inflammatory cells. The MMP family of enzymes contributes to both normal and pathological tissue. MMPs play a key role in the migration of normal and malignant cells. They also act as regulatory molecules, both by functioning in enzyme cascades and by processing matrix proteins, cytokines, growth factors and adhesion molecules to generate fragments with enhanced or reduced biological effects. The MMPs usually degrade multiple substrates, with considerable substrate overlap between individual MMPs. For example, interstitial collagenase (MMP-1) is capable of degrading casein, gelatin, antitrypsin, MBP, Selectin, pro-TNF and IL-1, and pro-MMP-2 and MMP-9. MMP-2 can degrade fibrillar collagen, elastin, IGF-binding proteins, FGF receptor and can activate MMP-1, MMP-9 and MMP-13. Collectively, they are capable of remodeling or degrading virtually all of the molecules of the extracellular matrix. This processing of the extracellular matrix occurs in wound healing and angiogenesis, as well as in development, differentiation, cell migration, and tumor cell metastasis. MMP-1 is important in wound healing because this metalloproteinase has been shown to play important roles in reepithelialization, formation of the provisional matrix, and angiogenesis. The triple helical structure of fibrillar collagen makes it very resistant to proteolysis, and only a very limited number of MMPs, including MMP-1, can cleave it.

MMPs are expressed as latent proenzymes, which must be activated by proteolytic cleavage of the pro-domain. A highly conserved cysteine at a constant position in the pro-domain, called the cysteine switch, functions in activation. This cysteine has been shown to coordinate with a zinc cation at the active site, thereby preventing hydration of the cation and subsequent proteolytic. Latent forms of MMPs can be activated by a variety of treatments affecting the cysteine. The present invention encompasses both the proenzyme form and the activated enzyme form of MMP-1, and fragments thereof.

In particular embodiments, the proteinase (such as MMP-1) is from a source heterologous to the cell type being cultured. E.g., in cell culture embodiments, the proteinase is not produced by cells in the medium, although it may have been produced by other cells. As used herein “not produced by cells in the medium,” or similar expressions, refers to proteinase that is in addition to any proteinase produced by cells or other substances in the medium; it does not refer to, e.g., a type of proteinase that is not produced by the cells, but simply to an external source of proteinase. Cells in the medium may, and often do, produce proteinase(s), some of which may be similar to or identical to the collagenase(s) useful in the invention; however, generally, in the context of cell culture, the proteinase of the invention is an externally added proteinase, whether or not it is a type similar to or identical to proteinase(s) produced by cells in the medium.

Some embodiments of the invention utilize a proteinase that is a matrix metalloprotease (MMP), where the MMP is not MMP produced by cells in the medium (i.e. is exogenous and/or heterologous to the cells in the medium). In some embodiments, the MMP is MMP-1, MMP-2, MMP-8, or MMP-9. In some embodiments, the MMP is mammalian MMP. In some embodiments, the mammalian MMP-1. Exemplary types include rat and human MMP-1. MMP-1 can degrade a broad range of substrates including types I, II, II, VII, VIII, and X collagens as well as casein, gelatin, alpha-1 antitrypsin, myelin basic protein, L-Selectin, pro-TNF, IL1b, IGF-BP3, IGF-BP5, pro MMP-2 and pro MMP-9. A significant role of MMP-1 is the degradation of fibrillar collagens in extracellular matrix remodeling, characterized by the cleavage of the interstitial collagen triple helix into ¾, ½ fragments. However, as the list of substrates above illustrates, the role of MMP-1 is more diverse than originally envisaged, and may involve enzyme cascades, cytokine regulation and cell surface molecule modulation. MMP-1 is expressed by fibroblasts, keratinocytes, endothelial cells, monocytes and macrophages. Structurally, MMP-1 may be divided into several distinct domains: a pro-domain which is cleaved upon activation, a catalytic domain containing the zinc binding site, a short hinge region and a carboxyl terminal (hemopexin-like) domain. See, e.g., “Interstitial Collagenase” by T. E. Cawston (2004) in Handbook of Proteolytic Enzymes (ed. A. J. Barrett, N. D. Rawlings, J. F. Woessner) pp. 472-480, Academic Press, San Diego, which is incorporated herein in its entirety.

Synthetic MMP-1 may be produced by peptide synthesis or as recombinant MMP-1. In some embodiments, recombinant human MMP-1 (rhMMP-1) is used. Such recombinant MMP-1 may be obtained from, e.g., R&D Systems, Minneapolis.

Matrix metalloprotease-inducing agents can also be useful in embodiments of the invention. These include IL-6, fibronectin fragments, and others known in the art.

The invention also encompasses compositions and methods that utilize functional variants, analogs, and other modifications of a collagenase, e.g., MMP-1, as well as peptide mimetics. As used herein, a “functional” variant, analog, fragment, modified polypeptide, peptide mimetic, and the like, encompasses a polypeptide or molecule that is a variant, analog, fragment, peptide mimetic or modified polypeptide of the native molecule (e.g., human MMP-1) that retains sufficient activity or function to produce the desired effect, either enhanced, unchanged, or decreased, when used in a composition or method of the invention.

Variants: Amino acid sequence variants of the proteinase, e.g., MMP-1, polypeptides of the present invention can be substitutional, insertional or deletion variants. Deletion variants lack one or more residues of the native protein that are not essential for function. Insertional variants typically involve the addition of material at a non-terminal point in the polypeptide. Terminal additions, called fusion proteins, are also encompassed by the invention.

The invention provides compositions useful in cell culture. These include cell culture media and additives for cell culture media. In some embodiments, the compositions contain a proteinase. “Exogenous proteinase” is used herein synonymously with “Proteinase not produced by cells in the medium,” and similar phrases, described elsewhere herein. “Heterologous proteinase, as well as similar phrases used herein, is a proteinase that is from a species that is different from the cells in the medium In some embodiments the compositions and methods utilize a collagenase, where the collagenase is not produced by cells in the culture medium and/or is from a species that is different than the cells in the culture medium. In some embodiments, the compositions and methods utilize a highly purified proteinase. In some embodiments, the composition and methods utilize a low-endotoxin proteinase. In some embodiments, the compositions contain an exogenous and/or heterologous MMP-1, e.g., a recombinant MMP-1 such as rhMMP-1. In some embodiments, the compositions contain a proteinase, which may be purified from tissue or cellular sources or produced by recombinant or other synthetic means, e.g., exogenous MMP-1. The compositions may further comprise other ingredients useful in the culture of cells or of a particular cell type.

a. Basal Cell Culture Media

A cell culture medium of the invention may be produced by addition of one or more ingredients to commercially available stock basal media, or it may be produced “from scratch,” i.e., by adding ingredients or groups of ingredients to a base such as distilled or deionized water.

When cell culture medium is produced by addition of ingredients to commercially available stock basal media, any of the numerous media available may be used. The basal medium employed, as known in the art, contains nutrients essential for supporting growth of the cell under culture, commonly including essential amino acids, fatty acids, and carbohydrates. The medium typically includes additional essential ingredients such as vitamins, cofactors, trace elements, and salts in assimilable quantities. Other biological compounds necessary for the survival/function of the particular cells, such as hormones and antibiotics may also be included. The medium also can include buffers, pH adjusters, pH indicators, and the like.

The choice of basal medium depends in part on the type of final medium desired, i.e., serum-containing, serum-free, animal protein-free, animal product-free, or defined. Exemplary useful media include all known suitable culture media and suitable culture media hereinafter developed which support maintenance and/or growth of the cells therein cultured.

A wide variety of commercially available basal media are available. Stock basal culture media of use in the invention include, but are not limited to, the following: Dulbecco's Modified Eagle Medium (“DMEM”), Iscove modified Dulbecco's' medium; DMEM/F-12 (1:1 DMEM and F-12 vol:vol); Medium 199; F-12 (Ham) Nutrient Mixture; F-10 (Ham) Nutrient Mixture; Minimal Essential Media (MEM), Williams' Media E; Fischer's or Waymouth's MB 752/1, CMRL, Puck's N15 Medium, Puck's N16 Medium; McCoy's 5A Medium, Leibo-vitz's L15 Medium; ATCC (American Type Culture Collection) CRCM 30; MCDB Media 101, 102, 103, 104; CMRL Media 1066, 1415, 1066, 1415; Roswell Park Memorial Institute Medium (RPMI) 1603, 1634, and 1640; and Hank's or Earl's Balanced Salt Solution. Several versions or modifications of many of these media are available, for example, DMEM 11966, DMEM 10314, MEM 11095, Williams' Media E 12251, Ham F12 11059, MEM-alpha 12561, and Medium-199 11151 (all available from Gibco-BRL/Life Technologies); and MCDB Media developed by Ham, such as MCDB 105, 110, 131, 151, 153, 201, and 302 media. In some embodiments, the basal medium employed is MCDB 153.

The above are merely exemplary, and it is understood that any stock basal medium, suitable for the cell type and application desired, may be used to produce the compositions of the invention. In addition, it will be realized that for optimal results, the basal medium to which the additional ingredient or ingredients is added must be appropriate for the cell type of interest, with key nutrients available at adequate levels to enhance cell growth or product expression. Thus, for example, it may be necessary to increase the level of glucose (or other energy source) in the basal medium, or to add glucose (or other energy source) during the course of culture, if this essential energy source is found to be depleted and to thus limit cell growth or product expression.

If the cell culture media are produced from scratch, standard techniques well-known in the art may be used. See, e.g., Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc.; Culture of Animal Cells—A Manual of Basic Technique, Ian Freshney, New York, Alan R. Liss, Inc., 1987), and U.S. Pat. Nos. 6,670,180; 6,048,728; 6,692,961; and 6,103,529, all of which are incorporated by reference herein.

If it is desired to produce a culture medium containing serum, a basal medium containing serum may be used, or serum may be added, or a medium may be made from scratch to include serum. One exemplary serum type commonly used in the art is fetal or new-born calf serum. Typically, serum contains substances that inhibit collagenases; thus, collagenase concentrations may need to be adjusted when serum is used compared to when the medium is serum-free. An exemplary inhibitor of collagenase found in serum, is alpha2-macroglobulin.

b. MMP-1

In embodiments where the proteinase is MMP-1, the MMP-1 is generally present in the medium according to the invention at a concentration sufficient to support the growth and/or viability of the cells, and to enhance the culture through, e.g., increasing the proportion of the cells in a primary culture that are the desired cell type, increasing population doublings, delaying senescence, or a combination of these. The exact concentration may vary depending on the cell type in use and the other media components present but may be easily determined using preliminary small-scale tests in accordance with conventional practice. Thus, for example, for any chosen medium, cells may be cultured on a small scale in the presence of a range of MMP-1 concentrations and the optimum concentration determined by observing the effect of different concentrations on cell growth and viability.

In some embodiments the proteinase is present in the medium at concentrations measured in units/ml (U/ml), wherein the activity is due to proteinase not produced by cells in the medium. The proteinase may be present at greater than about 0.000001, 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, or 3.0 U/ml. The proteinase may be present at less than about 0.00001, 0.001, 0.005, 0.01, 0.02, 0.03, 0.04, 0.05, 0.06, 0.07, 0.08, 0.09, 0.1, 0.2, 0.5, 1.0, 1.5, 2.0, 2.5, 3.0, 5.0, or 10.0 U/ml. E.g., the collagenase may be present at about 0.00001-0.1, 0.00005-0.5, 0.0001-0.1, or 0.0001-0.05 U/ml.

In some embodiments utilizing highly pure MMP-1, e.g., rhMMP-1, MMP-1 is present in the culture medium at a concentration from about 0.01 ng/ml to about 100 ng/ml, or about 0.05 ng/ml to about 50 ng/ml, or about 0.1 ng/ml to about 20 ng/ml, or about 0.1 ng/ml to about 10 ng/ml, or about 0.5 ng/ml to about 5.0 ng/ml, or about 0.8-3 ng/ml, or about 1.5-2.0 ng/ml, or about 1-3 ng/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ng/ml. In some embodiments less highly purified proteinase may be used, e.g., crude proteinase from Clostridium histolyticum which, in some embodiments, has been partially purified, e.g., to remove endotoxins. Such proteinase preparations may be present at a concentration of about 0.02 ng/ml to about 200 ng/ml, or about 0.1 ng/ml to about 100 ng/ml, or about 0.2 ng/ml to about 40 ng/ml, or about 0.2 ng/ml to about 20 ng/ml, or about 1.0 ng/ml to about 10 ng/ml, or about 1-7 ng/ml, or about 2-3.5 ng/ml, or about 0.5, 1, 1.5, 2, 2.5, 3, 3.5 4, 4.5, 5, 5.5, 6, 6.5, 7, 7.5, 8, 8.5, 9, 9.5, 10, or more than 10 ng/ml.

The cell culture medium may be used for the culture of any suitable cell type. In some embodiments, the cell culture medium or supplement containing a proteinase, is intended for use with a cell type selected from the group consisting of myocytes, fibroblasts, osteoblasts, chondrocytes, Schwann cells, neurons, hepatocytes, cardiomyocytes, adipocytes, and myocytes. In some embodiments, the cells are derived from an organism from the phylum Chordata. In some embodiments, the cells are derived from an organism from the class Aves. In some embodiments, the cells are derived from an organism of the class Mammalia. In some embodiments, the cells are derived from an organism of the class Chondrichthyes or the class Osteichthyes. In some embodiments, the cells are derived from an organism of the class Amphibia. In other embodiments, the cells are derived from an organism of the kingdom Fungi. In some embodiments, the cells are derived from an organism of the division Ascomycota. In some embodiments, the cells are derived from an organism of the class Saccaromycetes.

c. Additional Ingredients

The compositions of the invention may include additional ingredients useful in the culture of a particular cell type or of cells in general.

In cell culture medium that is serum-containing, serum can be added if not already present in the basal cell culture medium, or if the cell culture medium is produced from scratch. Serum contains a number of biochemical entities that the cells need for survival. Some of these entities protect the cells against toxic impurities, some of which may be products of the cultured cells themselves, and others serve to present iron and trace metals to the cells in a way the cells can use. The addition of serum can produce a well-functioning medium for many different cell types. The serum should be pathogen free and carefully screened for mycoplasma bacterial, fungal, and viral contamination. High-quality FBS needed for the reproducibility of scientific research, can come from any country of origin, as long as regulatory and industry standards are adhered to. Serum for cell culture is generally fetal calf serum (FCS) or newborn calf serum. While FCS is the most commonly applied supplement in animal cell culture media, other serum sources are also routinely used, including newborn calf, horse and human.

In cell culture medium that is animal product-containing, extracts from organs or glands may also be used for the supplementation of culture media. These include extracts of submaxillary gland (see, e.g., Cohen, S., J. Biol. Chem. 237:1555-1565 (1961)), pituitary (see, e.g., Peehl, D. M., and Ham, R. G., In Vitro 16:516-525 (1980); U.S. Pat. No. 4,673,649), hypothalamus (see, e.g., Maciag, T., et al., Proc. Natl. Acad. Sci. USA 76:5674-5678 (1979); Gilchrest, B. A., et al., J. Cell. Physiol. 120:377-383 (1984)), ocular retina (see, e.g., Barretault, D., et al., Differentiation 18:29-42 (1981)) and brain (see, e.g., Maciag, T., et al., Science 211:1452-1454 (1981)). These types of chemically undefined supplements serve several useful functions in cell culture media. In some embodiments, the cell culture medium or supplement contains bovine pituitary extract (BPE).

A serum-free culture medium may contain BPE at an appropriate concentration for the growth of the cells for which it is intended. For animal protein-free, animal product-free, or defined media, BPE is not appropriate. Alternatively, an admixture of heparin, epidermal growth factor (EGF), a cAMP-increasing agent(s) and fibroblast growth factor(s) (FGF(s)) may be used as a replacement for BPE or other organ/gland extracts in animal cell culture media. If the source of the proteins is not of animal origin (e.g., if the proteins are recombinantly produced), then such an admixture may be appropriate for an animal product-free or animal protein-free medium. See below for a further description of these ingredients. See also U.S. Pat. No. 6,692,961, the disclosure of which is incorporated herein by reference.

Growth factors may be added to the cell culture medium or supplement. An example of a growth factor of use in the compositions of the invention is epidermal growth factor (EGF). EGF may be natural or recombinant and may be, e.g., human or rodent. EGF is available commercially (e.g., from GIBCO/LTI, Gaithersburg, Md.), or may be isolated from natural sources or produced by recombinant DNA techniques (U.S. Pat. No. 4,743,679) according to methodologies that are routine in the art. EGF can be added to the cell culture medium at a concentration of about 0.01-10,000 ng/ml, or about 0.1-0-100 ng/ml, or about 0.002-20 ng/ml, or about 0.02-2 ng/ml, or about 0.04-1 ng/ml, or about 0.08-0.8, or about 0.05, 0.1, 0.2, 0.3, 0.4, or 0.5 ng/ml. In some embodiments, EGF is present at about 0.2 ng/ml.

In addition, any of the fibroblast growth factor (FGF) family may be used, including FGF-1 (acidic FGF or aFGF), FGF-2 (basic FGF or bFGF), FGF-3 (int-2), FGF4 (K-FGF), FGF-5 (hst-1), FGF-6 (hst-2) and FGF-7 (myocyte growth factor or MGF). Natural or recombinant FGF may be used, which may be of human, bovine, porcine or rodent origin. For example, recombinant human aFGF may be used. aFGF, bFGF and KGF are available commercially (e.g., from GIBCO/LTI, Gaithersburg, Md. and R&D Systems, Inc., Minneapolis, Minn.), or may be isolated from natural sources or produced by recombinant DNA techniques (EP 0 408 146 and U.S. Pat. No. 5,395,756 for aFGF; U.S. Pat. No. 5,189,148 for bFGF; WO 90/08771 and WO 95/01434 for KGF) according to methodologies that are routine in the art. FGF can be added to the medium to a concentration of about 0.1-10,000 ng/ml, or about 1-100 ng/ml, or about 1-10 ng/ml, or about 1, 2, 3, 4, 5, 6, 7, 8, 9, or 10 ng/ml. In some embodiments FGF-2 is present at about 5 ng/ml. Other growth factors that may be added include HGF, heregulin, NGF, or other growth factors depending on the cell type to be cultured.

Other ingredients useful in cell culture media or supplements of the invention include Extracellular matrix proteins, insulin, transferrin, hydrocortisone, and heparin. Insulin may be present at a concentration of about 0.05-500 ug/ml, or about 0.5-50 ug/ml, or about 1-25 ug/ml, or about 2-15 ug/ml, or about 1, 2, 3, 4, 5, 6, 7, 8, 9, or 10 ug/ml. In some embodiments, insulin is present at 5 ug/ml. Transferrin may be present at a concentration of about 0.1-10,000 ug/ml, or about 1-100 ug/ml, or about 2-20 ug/ml, or about 5-15 ug/ml, or about 5, 6, 7, 8, 9, 10, 11, 12, 13, 14, or 15 ug/ml. Hydrocortisone may be present at a concentration of about 0.001-10 ug/ml, or about 0.01-1 ug/ml, or about 0.02-0.5 ug/ml, or about 0.1-0.2 ug/ml. Heparin may be obtained commercially, for example from Sigma (Saint Louis, Mo.), and is derived, e.g., from porcine mucosa. Heparin is added to the present media primarily to stabilize the activity of the growth factor components, especially FGF. To formulate the medium of the present invention, heparin is added at a concentration of about 1-500 U.S.P. units/liter, or about 5-50 U.S.P. units/liter, or about 5-20 USP/L, or about 10-15 USP/L.

Ascorbic acid may also be added to the medium. Ascorbic acid is available commercially in several forms. An exemplary ascorbic acid for use in formulating the present media is L-ascorbic acid phosphate, magnesium salt, available from Wako Pure Chemical Industries. Ascorbic acid can be added to the medium at a concentration of about 0.001-10 mg/L, or about 0.01-5 mg/L. In some embodiments, ascorbic acid is present at a concentration of about 0.1 mg/L.

Further additional ingredients for culture media of the invention include purines, glutathione monobasic sodium phosphates, sugars, deoxyribose, ribose, nucleosides, lipids, acetate salts, phosphate salts, HEPES, phenol red, pyruvate salts and buffers. Other ingredients often used in media include steroids and their derivatives, cholesterol, fatty acids and lipids, Tween 80, 2-mercaptoethanol, pyrimidines antibiotics (gentamicin, penicillin, streptomycin, amphotericin B, etc.) whole egg ultra-filtrate, and attachment factors (fibronectins, vitronectins, collagens, laminins, tenascins, etc.). The concentrations of the ingredients are well known to one of ordinary skill in the art.

For cell culture media made from scratch, or for additional supplementation of conventional basal media, the ingredients are well-known in the art. Such ingredients are useful when the medium is, e.g., a defined medium. See, e.g., Cell Culture Methods for Molecular and Cell Biology, Vol. 1: Methods for Preparation of Media, Supplements, and Substrata for Serum-Free Animal Cell Culture, Barnes, D. W., et al., eds., New York: Alan R. Liss, Inc.; Culture of Animal Cells—A Manual of Basic Technique, Ian Freshney, New York, Alan R. Liss, Inc., (1987); Culture of Epithelial Cells, Freshney, R. I., ed., New York: Wiley-Liss, (1992); Animal Cell Biotechnology, Vol. 1, Spier, R. E. et al., Eds., Academic Press New York (1985); Ham, R. G., Methods for Preparation of Media, Supplements and Substrata for Serum-Free Animal Culture, Alan R. Liss, Inc., New York (1984)) Waymouth, C., Methods for Preparation of Media, Supplements and Substrata for Serum-Free Animal Culture, Alan R. Liss, Inc., New York (1984); Cell & Tissue Culture: Laboratory Procedures, John Wiley & Sons Ltd., Chichester, England 1996; and U.S. Pat. Nos. 6,833,271; 6,692,961; 6,593,140; 6,103,529; 6,323,025; and 6,048,728, all of which are incorporated by reference herein in their entirety.

Briefly, for media made from scratch, or for supplementation of conventional basal media, ingredients may include amino acids, vitamins, inorganic salts, adenine, ethanolamine, D-glucose, heparin (mentioned above), N-[2-hydroxyethyl]piperazine-N′-[2-ethanesulfonic acid](HEPES), hydrocortisone (mentioned above), insulin (mentioned above), lipoic acid, phenol red, phosphoethanolamine, putrescine, sodium pyruvate, triiodothyronine (T3), thymidine and transferrin (mentioned above). Alternatively, insulin and transferrin may be replaced by ferric citrate or ferrous sulfate chelates. Each of these ingredients may be obtained commercially, for example from Sigma (Saint Louis, Mo.).

Amino acid ingredients which may be included in the media of the present invention include L-alanine, L-arginine, L-asparagine, L-aspartic acid, L-cysteine, L-glutamic acid, L-glutamine, glycine, L-histidine, L-isoleucine, L-leucine, L-lysine, L-methionine, L-phenylalanine, L-proline, L-serine, L-threonine, L-tryptophan, L-tyrosine and L-valine. These amino acids may be obtained commercially, for example from Sigma (Saint Louis, Mo.). In some embodiments it may be useful to include the D-form of any of the amino acids listed above, with the exception of glycine.

Vitamin ingredients which may be included in the media of the present invention include biotin, choline chloride, D-Ca⁺⁺-pantothenate, folic acid, i-inositol, niacinamide, pyridoxine, riboflavin, thiamine and vitamin B₁₂. Formulations may also include fat soluble vitamins (including A, D, E and K). Vitamins may be obtained commercially, for example from Sigma (Saint Louis, Mo.).

Inorganic salt ingredients which may be used in the media of the present invention include a calcium salt (e.g., CaCl₂)), CuSO₄, FeSO₄, KCl, a magnesium salt (e.g., MgCl₂), a manganese salt (e.g., MnCl₂), Sodium acetate, NaCl, NaHCO₃, Na₂HPO₄, Na₂SO₄ and ions of the trace elements selenium, silicon, molybdenum, vanadium, nickel, tin and zinc. These trace elements may be provided in a variety of forms, preferably in the form of salts such as Na₂SeO₃, Na₂SiO₃, (NH₄)₆Mo₇O₂₄, NH₄VO₃, NiSO₄, SnCl and ZnSO₄. These inorganic salts and trace elements may be obtained commercially, for example from Sigma (Saint Louis, Mo.).

If the medium is made from scratch, the medium ingredients can be dissolved in a liquid carrier. The pH of the medium typically is adjusted to about 7.0-7.6, or about 7.1-7.5, or about 7.2-7.4. The osmolarity of the medium typically is adjusted to about 275-350 mOsm, or about 285-325 mOsm, or about 280-310 mOsm. The type of liquid carrier and the method used to dissolve the ingredients into solution vary and can be determined by one of ordinary skill in the art with no more than routine experimentation. Generally, the medium ingredients can be added in any order.

The culture media or additives of the present invention can be sterilized to prevent unwanted contamination. Sterilization may be accomplished, for example, by filtration through a low protein-binding membrane filter of about 0.1-1.0 um pore size (available commercially, for example, from Millipore, Bedford, Mass.) after admixing the concentrated ingredients, to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1×sterile medium formulation. Autoclaving or other elevated temperature-based methods of sterilization are not favored, since many of the components of the present culture media are heat labile and will be irreversibly degraded by temperatures such as those achieved during most heat sterilization methods.

d. Concentrated Solutions and Additives

In addition to providing culture media that are complete and ready-to-use, the invention also provides concentrated media, and additives for addition to conventional basal media to enhance cell growth, purity, and/or viability. For concentrated media, or for additives for addition to basal media, the solutions containing ingredients are more concentrated than the concentration of the same ingredients in a 1× media formulation. The ingredients can be 10-fold more concentrated (10× formulation), 25-fold more concentrated (25× formulation), 50-fold more concentrated (50× formulation), or 100-fold more concentrated (100× formulation). More highly concentrated formulations can be made, provided that the ingredients remain soluble and stable. See, e.g., U.S. Pat. No. 5,474,931, which is directed to methods of solubilizing culture media components at high concentrations. In some embodiments, various components of a medium are supplied at different concentration levels.

Media or supplements of the invention may also be provided as a lyophilized powder. As will be apparent, the proper amount of a proteinase and/or other components to produce the proper final concentration when admixed with a predetermined volume of culture medium or other appropriate diluent may be supplied in appropriate packaging.

If the media or additives are prepared as separate concentrated solutions, or as lyophilized powders, an appropriate (sufficient) amount of each concentrate is combined with a diluent to produce a 1×medium formulation. The diluent used can water or other solutions including aqueous buffers, aqueous saline solution, or other aqueous solutions. In some embodiments the diluent is a convention basal or cell culture medium, to which an additive of the invention is added in concentrated or lyophilized form.

In some embodiments, the cell culture medium or supplement is packaged for transport, storage and/or use by a consumer. Such packaging of tissue culture medium for transport, storage, and/or use is well-known in the art. Packaged medium may include further components for the dispensing and storage of the medium and may also include separately packaged diluent for dilution of concentrated medium, optional additional ingredients for inclusion by the user if desired, instructions for use, and the like.

### 2. Methods

In one aspect, the invention provides methods of culturing cells. Cells are cultured in media that contain a proteinase, e.g., exogenous MMP-1. The media may also contain an exogenous cAMP-elevating agent. Other ingredients, as described for the production of cell culture media, may also be included.

a. Cell Types that May be Cultured

The compositions and methods of the invention are suitable for the culture of a variety of cells, especially eukaryotic cells. The media of the invention are suitable for culturing animal cells, especially mammalian cells; plant cells; insect cells; arachnid cells; and microorganisms such as bacteria, fungi, molds, protozoa, and rickettsia, including antibiotic-producing cells. Exemplary applications include the culture of cloned cells, such as hybridoma cell lines; of mammalian cells for the production of cell products, especially proteins and peptides such as hormones, enzymes, and immunofactors; of virally-infected cells for the production of vaccines; of plant cells in, for example, meristem or callus culture; of epithelial cells to provide tissue for wound healing; of resistant cells for medical and diagnostic use; and in media adapted for the production and preservation of biological organs and implant tissue.

Specific cell types useful for culture in the processes of the invention accordingly include: cells derived from mammalian tissues, organs and glands such as the brain, heart, lung, skeletal muscle, stomach, intestines, thyroid, adrenal, thymus, parathyroid, testes, liver, kidney, bladder, spleen, pancreas, gall bladder, ovaries, uterus, prostate, and skin; reproductive cells (sperm and ova); lymph nodes, bone, cartilage, and interstitial cells; blood cells including immunocytes, cytophages such as macrophages, lymphocytes, leukocytes, erythrocytes, and platelets. Additional cell types include stem, leaf, pollen, and ovarian cells of plants; microorganisms and viruses as specified above; and cells derived from insect or arachnid tissues, organs, and glands.

Mammalian and other cells particularly suitable for cultivation in the present media include those of human origin, which may be primary cells derived from a tissue sample, e.g. Stem cells (Human and mouse; Adult and Embryonic), Chicken Egg Fibroblasts, Microglia, Human and monkey skeletal muscle cells, Mast cells, Macrophages Eosinophils Human endothelial cells, Shwann cells Hippocampal neurons Astrocytes Monocytes Dorsal root ganglion, Neurons, Adipocytes, Kidney cells, Melanoma cells, Embryonic fibroblasts, Pancreatic beta cells, Beta islet, Embryonic cardiomyocytes, Intestinal epithelial cells, Hepatocytes, Bone marrow, T cells, Human Corneal Epithelial Model, Blood Brain Barrier, Bladder Cell, Endothelial Cell, Melanocyte Cell, Mammary Epithelial Cell, Smooth Muscle Cell, Skeletal Muscle Cell, Neural Cell, Prostate Cell, Renal Cell, Renal Epithelial Cell, Skeletal Cell, Lymphocytes, Monocytes, Bone Marrow, Peripheral Blood, Glial, Embryonic Stem Cell Mice, hematopoietic cells, Monkey Kidney Cells, Synovial Cells, and HUVEC. Further examples of mammalian cells include diploid cell strains such as MRC-5 and WI-38, trans-formed cells or established cell lines (e.g., HeLa), each of which may optionally be diseased or genetically altered. Other mammalian cells, such as hybridomas, CHO cells, COS cells (e.g., COS-7L), VERO cells (monkey kidney epithelial cells), HeLa cells, 293 cells (embryonal human kidney), rabbit kidney cells, PER-C6 cells, K562 cells, MOLT-4 cells, M1 cells, NS-1 cells, COS-7 cells, MDBK cells, MDCK cells, MRC-5 cells, WI-38 cells, WEHI cells, SP2/0 cells, BHK cells (including BHK-21 cells) and derivatives thereof, are also suitable for cultivation in the present media. In particular, stem cells and cells used in vitro virus production may be cultivated in the media of the present invention. Tissues, organs, organ systems and organisms derived from animals or constructed in vitro or in vivo using methods routine in the art may similarly be cultivated in the culture media of the present invention.

Primary culture of cell types of the invention includes, in some embodiments, culture of cells such as fibroblasts, osteoblasts, chondrocytes, Schwann cells, neurons, cardiomyocytes, hepatocytes, myoblasts, adipocytes and endothelial cells.

The compositions and methods of the invention are also useful for culturing epithelial cells, e.g., keratinocytes. Successful culture of keratinocytes has proven to be difficult, owing primarily to their nutritional fastidiousness. Keratinocytes from skin are often rapidly over-grown by less fastidious and faster-growing fibroblasts that were also resident in the tissue. This is especially true in the culture of fetal keratinocytes, because, unlike in adult or neonatal skin, it is generally not possible to separate dermis from epidermis in fetal skin, and thus the dermal fibroblasts are present along with the epidermal keratinocytes in the initial culture.

b. Methods of Cell Culture

Animal cells for culturing by the present invention may be obtained commercially, for example from ATCC (Rockville, Md.), Cell Systems, Inc. (Kirkland, Wash.) or Thermo-Fisher Scientific (San Diego, Calif.). Alternatively, cells may be isolated directly from samples of animal tissue obtained via biopsy, autopsy, donation or other surgical or medical procedure.

Generally, tissue should be handled using standard sterile technique and a laminar flow safety cabinet. In the use and processing of all human tissue, the recommendations of the U.S. Department of Health and Human Services/Centers for Disease Control and Prevention typically should be followed (Biosafety in Microbiological and Biomedical Laboratories, Richmond, J. Y. et al., Eds., U.S. Government Printing Office, Washington, D.C. 3rd Edition (1993)). The tissue is cut into small pieces (e.g., 0.5×0.5 cm) using sterile surgical instruments. The small pieces are washed twice with sterile saline solution supplemented with antibiotics, and then may be optionally treated with an enzymatic solution (e.g., collagenase or trypsin solutions, each available commercially, for example, from Thermo-Fisher Scientific., Rockville, Md.) to promote dissociation of cells from the tissue matrix.

Cells may be isolated by any technique known or developed in the art. In one typical technique, the mixture of dissociated cells and matrix molecules are washed twice with a suitable physiological saline or tissue culture medium (e.g., Dulbecco's Phosphate Buffered Saline without calcium and magnesium). Between washes, the cells are centrifuged (e.g., at 200×g) and then resuspended in serum-free tissue culture medium. Aliquots may be counted using an electronic cell counter (such as a Coulter Counter). Alternatively, the cells can be counted manually using a hemocytometer.

The isolated cells can be plated according to the experimental conditions determined by the investigator. The examples below demonstrate at least one functional set of culture conditions useful for cultivation of certain mammalian cells. It is to be understood, however, that the optimal plating and culture conditions for a given animal cell type can be determined by one of ordinary skill in the art using only routine experimentation. For routine culture conditions, using the present invention, cells can be plated onto the surface of culture vessels without attachment factors. Alternatively, the vessels can be precoated with natural, recombinant or synthetic attachment factors or peptide fragments (e.g., collagen or fibronectin, or natural or synthetic fragments thereof).

Isolated cells can also be seeded into or onto a natural or synthetic three-dimensional support matrix such as a preformed collagen gel or a synthetic biopolymeric material, or onto feeder layers of cells. Use of attachment factors or a support matrix with the medium of the present invention will enhance cultivation of many attachment-dependent cells in the absence of serum supplementation. Thus, culture techniques useful in the methods of the invention include the use of solid supports, (especially for anchorage-dependent cells in, for example, mono-layer or suspension culture) such as glass, carbon, cellulose, hollow fiber membranes, suspendable particulate membranes, and solid substrate forms, such as agarose gels. In the latter embodiments, it is possible to entrap the collagenase, e.g., MMP-1, e.g., it can be caged within the bead, trapped within the matrix, or covalently attached, i.e. as a mixed disulfide.

The cell seeding densities for each experimental condition can be optimized for the specific culture conditions being used. For routine culture in plastic culture vessels, an initial seeding density of 0.1-1.0×10⁵ cells per cm² or about 1.5× the plating concentration routinely used for the same cells in serum supplemented media is preferable.

Mammalian cells are typically cultivated in a cell incubator at about 37° C., while the optimal temperatures for cultivation of fish, aquatic species, avian, nematode and insect cells are typically somewhat lower and are well-known to those of ordinary skill in the art. The incubator atmosphere should be humidified for cultivation of animal cells and should contain about 3-10% carbon dioxide in air. Culture medium pH should be in the range of about 7.1-7.6, in some embodiments about 7.1-7.4, and in some embodiments about 7.1-7.3.

Cells in closed or batch culture should undergo complete medium exchange (i.e., replacing spent media with fresh media) about every 2-3 days, or more or less frequently as required by the specific cell type. Cells in perfusion culture (e.g., in bioreactors or fermenters) will receive fresh media on a continuously recirculating basis. In particular aspects the method comprise culturing the cells in the culture medium for at least one day. Further embodiments include culturing the cells in the culture medium for at least two days. More particular aspects include culturing the cells in the culture medium for at least five days or longer.

The methods of the invention include culturing cells in a medium that contains an proteinase e.g., exogenous MMP-1. In some embodiments, the methods of the invention include culturing cells in a medium that contains a proteinase. e.g., exogenous MMP-1. The methods of the invention are useful in primary cultures; serial cultures; subcultures; preservation of cultures, such as frozen or dried cultures; and encapsulated cells; cultures also may be transferred from conventional media to media containing a proteinase by known transfer techniques.

According to the practice of the invention, cells are exposed to proteinase, e.g., MMP-1, in amounts sufficient to promote culture of these cells in vitro, as measured, for example, by significant decrease in doubling time, increase in purity of the cell culture for the cell type of interest, increase in cell lifespan, increase in cell viability, increase in cell biomass, in-crease in cell bioproductivity, delay of cell senescence, or diversification or normalization of cell function as compared to unexposed cells.

In certain embodiments of the subject method, it will be desirable to monitor the growth state of cells in the culture, e.g., cell proliferation, differentiation and/or cell death. Methods of measuring cell proliferation are well known in the art and most commonly include determining DNA synthesis characteristic of cell replication. There are numerous methods in the art for measuring DNA synthesis, any of which may be used according to the invention. For example, DNA synthesis can be determined using a radioactive label (³H-thymidine) or labeled nucleotide analogues (BrdU) for detection by immunofluorescence. Cell nuclei that have incorporated BrdU during DNA synthesis can be identified using mouse monoclonal anti-BrdU (Dako; Carpintaria, Calif.), detected with the immuno-peroxide technique of Stemberger et al., J. Histochem., Cytochem. 18: 315 (1970), followed by hematoxylin counterstaining.

Included herein are methods of optimizing the concentration of proteinases and/or the concentration of growth factor to minimize (reduce) the doubling time of the cells being grown and propagated. Such optimization may be performed using any of the compositions or methods described herein. The optimization of the concentrations of proteinases and growth factors leads to reductions in the concentration of the growth factors. In comparing the concentration of growth factors in media containing proteinase to media lacking proteinase, that are otherwise identical, the concentration of growth factors in the media lacking proteinase will be higher. In some embodiments, the concentration of growth factor in the media lacking proteinase is between about 1.25 fold and 200 fold higher. In some embodiments, the concentration of growth factor in the media lacking proteinase is between about 10 fold and 160 fold higher. In some embodiments, the concentration of growth factor in the media lacking proteinase is between about 2 fold and 120 fold higher. In some embodiments, the concentration of growth factor in the media lacking proteinase is 2 fold higher. In some embodiments, the concentration of growth factor in the media lacking proteinase is 3 fold higher. In some embodiments, the concentration of growth factor in the media lacking proteinase is 4 fold higher. In some embodiments, the concentration of growth factor in the media lacking proteinase is 5 fold higher. In some embodiments, the concentration of growth factor in the media lacking proteinase is 10 fold higher. In some embodiments, the concentration of growth factor in the media lacking proteinase is 20 fold higher. In some embodiments, the concentration of growth factor in the media lacking proteinase is 30 fold higher. In some embodiments, the concentration of growth factor in the media lacking proteinase is 40 fold higher. In some embodiments, the concentration of growth factor in the media lacking proteinase is 50 fold higher. In some embodiments, the concentration of growth factor in the media lacking proteinase is 60 fold higher. In some embodiments, the concentration of growth factor in the media lacking proteinase is 70 fold higher. In some embodiments, the concentration of growth factor in the media lacking proteinase is 80 fold higher. In some embodiments, the concentration of growth factor in the media lacking proteinase is 90 fold higher. In some embodiments, the concentration of growth factor in the media lacking proteinase is 100 fold higher.

Particular methods further comprise optimizing the concentration of the proteinase to achieve the lowest double time for the cells. Methods may further comprise optimizing the concentration of the growth factor to the lowest concentration that supports the shortest doubling time for the cells.

c. Methods of Cell Culture to Produce an Animal Cell Product

The invention also provides methods of cell culture to produce an animal cell product, where the cell culture is exposed at some point during culture of the cells to proteinase e.g., MMP-1. Thus, media according to the invention may be used to culture animal cells to obtain an animal cell product. In some embodiments, the invention provides a process for obtaining an animal cell product by cell culture which comprises the steps of (1) culturing animal cells which produce said product in a nutrient culture medium comprising assimilable sources of carbon, nitrogen, amino acids, iron and other inorganic ions, trace elements and optionally lipids and growth promoters or regulators in admixture with a proteinase, e.g., MMP-1, (2) continuing the culture until said product accumulates and (3) recovering said product.

Cell products which may be obtained according to the invention include any products that are produced by cultured animal cells. Typical products include polypeptides and proteins, for example immunoglobulins such as monoclonal and recombinant antibodies and fragments thereof, hormones such as erythropoietin and growth hormone, e.g. human growth hormone, lymphokines such as interferon, interleukins such as interleukin 2, 4, 5 and 6 and industrially and therapeutically useful enzymes such as tissue plasminogen activator.

In the process according to the invention, the animal cells may generally be cultured in suspension in the culture medium in a suitable culture vessel, for example a stirred tank or airlift fermenter, using known culture techniques. The production of the desired products during the culture may be monitored using any appropriate assay for the particular product in question. Thus, for example, where the product is a polypeptide or protein, the production of this may be monitored by general assay techniques such as enzyme-linked immunoabsorbent assay or immunoradiometric assay adapted for use with the particular polypeptide or protein.

Where in the process according to the invention it is desired to isolate the cell product obtained, this may be achieved using conventional separation and purification techniques. Thus, for example, where the product is secreted by the cells into the medium it may be separated from the cells using techniques such as centrifugation and filtration and then further purified using, for example, affinity purification techniques, such as affinity chromatography. Where the product is not secreted by the cells, the above methods may still be used, but after the cells have first been lysed to release the product

### D. Myocyte, Adipocyte and Fibroblast Culture

### 1. General

In certain embodiments the present invention provides methods useful in myocyte, adipocyte, and fibroblasts culture systems where the cells are exposed to proteinase e.g., MMP-1. In some embodiments, the methods of the invention are used in the culture of myocytes adipocyte and fibroblasts in serum-free culture systems or animal product-free culture systems. The methods of the invention are particularly useful for cultivating myocytes, adipocyte and fibroblasts.

### 2. Methods

Any source of myocytes, adipocyte and fibroblasts may be used in the methods of the invention. The myocyte, adipocyte and fibroblasts may be of animal or human origin, and may be from fetal, newborn, juvenile, or adult organisms.

Typically, the initial source of myocytes and fibroblasts is skeletal muscle. Skeletal muscle can be obtained by appropriate biopsy or upon autopsy. In the case of animal muscle, the animal may be sacrificed, and muscle removed and treated after sacrifice. In some embodiments, the tissue (muscle) is cleaned, removed and placed in appropriate medium, e.g., Dulbecco's Modified Eagle's Medium (DMEM). Other media may be used, as will be apparent to those of skill in the art.

The cells may be cultured in any manner known in the art including in monolayer, beads or in three-dimensions and by any means (i.e., culture dish, roller bottle, a continuous flow system, etc.). Methods of cell and tissue culturing are well known in the art, and are described, for example, in Cell & Tissue Culture: Laboratory Procedures, John Wiley & Sons Ltd., Chichester, England 1996; Freshney, Culture of Animal Cells: A Manual of Basic Technique, 2d Ed., A. R. Liss, Inc., New York, 1987, both of which are incorporated herein by reference in their entirety.

The cell culture medium may be any myocyte, myocyte, adipocyte and fibroblast culture medium described herein. In addition, the initial culture of the cells may be in conventional medium, without any proteinase; however, it is preferable to begin culturing the cells in medium of the invention that includes proteinases, as described herein. In most embodiments of the invention, the culture medium is serum-free. In some embodiments of the invention, the medium is serum-containing. In some embodiments, the medium is animal product-free. In some embodiments, the medium is animal protein-free. The medium may be used with or without extracellular matrix proteins (ECM). The medium may be used with or without bovine pituitary extract (BPE). Although some cultures are improved with BPE, BPE adds unknown factors from a bovine source. It is not necessary for myocytes, adipocytes and/or and fibroblasts culture according to the methods of the invention.

The medium is replaced at intervals. The intervals may be regular or irregular. Replacement intervals can be from about 0.25 to about 4 days, or about 0.5 to about 2 days, or about 1 day, about 2 days, about 3 days, about 4 days, about 5 days, and about 6 days. In some embodiments, the medium is replaced weekly.

The media may be replaced for about 1, 2, 3, 4, 5, 6, 7, 8, 9, 10, or more than about 10 days. In some embodiments, the media is replaced for about 4, about 5, or about 6 days. In some embodiments, the media is replaced for about 5 days.

Thus, in some embodiments, the methods of the invention comprise the production of cultures of myocytes, adipocytes and fibroblasts that comprise at least about 60, 70, 80, 90, 95, 98, 99, 99.5, 99.9, or 99.99% myocytes, adipocytes and fibroblasts by culturing the myocytes, adipocytes and fibroblasts in the presence of proteinase. In some embodiments, the methods of the invention comprise the production of cultures of myocytes that comprise between about 90 and 99.99% myocytes, or between about 95 and 99.99% myocytes, or between about 98 and 99.99% myocytes, or between about 99 and 99.99% myocytes by culturing the myocytes in the presence of proteinase.

Cells are passed when about 60-70% confluent. It is important for tissue culture not to let the cells grow to or near confluence. In some cases, it may be wished to grow cells to greater degrees of confluence than 60-70% in order to obtain medium for certain therapeutic or other uses.

Cell lines can be passaged indefinitely, where the cells are cultured for part or all of the culture period in medium that contains proteinase.

### Example 1

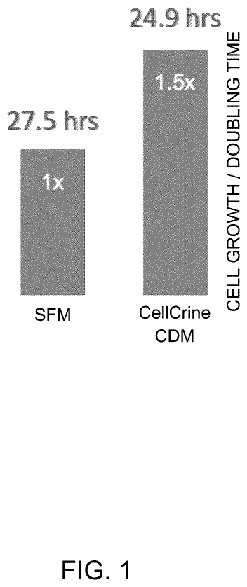

The cell culture media discussed above has been utilized to grow several different types of cells in one example, as seen in FIG. 1, the media was tested growing chicken myocytes in serum free media without the proteinase and then with the proteinase. The serum free media was composed of growth factors, water, salts, amino acids, vitamins, glucose, heregulin, selenium, transferrin, insulin, ascorbic acid, and without proteinase. Each in an amount sufficient to keep cells undifferentiated, support survival, and promote proliferation of somatic and stem cells. This media was compared to media composed of water, salts, amino acids, vitamins, glucose, growth factors heregulin, selenium, transferrin, insulin, ascorbic acid, enzymes, and each in an amount sufficient to keep cells undifferentiated, support survival, and promote proliferation of somatic and stem cells. In this example the concentration of the proteinase, in this case highly purified collagenase, in the media is 5 ng/ml and the concentration of the growth factor, in this case FGF-2 is 1 ng/ml. Standard protocols indicate that the amount of growth factor used in the growth of cells is 40-100 ng/ml. Accordingly, the reduction of the amount of growth factor is between 40 and 100 times. The cell growth between the two different culture media types was normalized to 1. The serum free media had a doubling time at 1× of 27.5 hours. In contrast, the doubling time with the proteinase dropped to 24.9 hours and a 1.5×. This shows a significant improvement in growth for the use of the proteinase and reduced growth factors. This improved cell efficiency leads to higher cell yields in cultured cells.

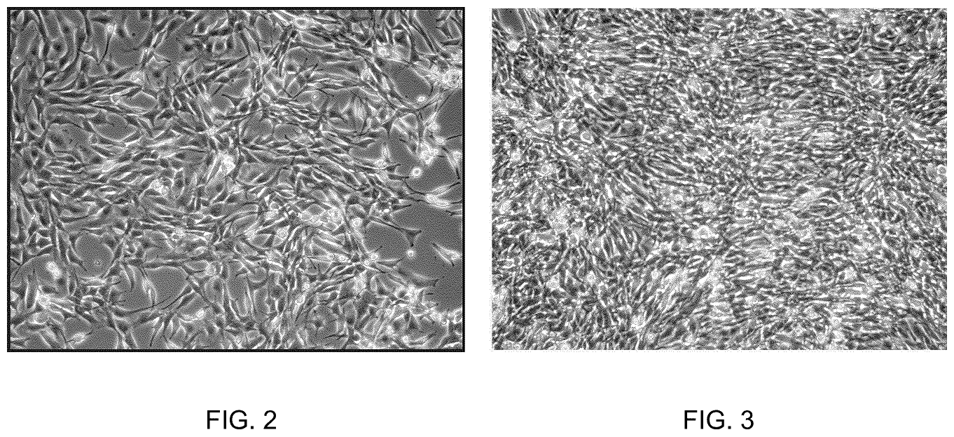

FIG. 2 shows the growth of the cells in the serum free medium without the proteinase As can be seen on this micrograph, the cells retain space around them and cannot be grown as tightly packed together.

As can be seen in FIG. 3 which is a micrograph of cells grown in the culture media which contains the proteinase, the cells are able to grow more closely together, thus more cells are able to be grown in the same space than can be grown in culture using media without the proteinase. The denser cells shows the increased cellular efficiency and the higher yield for the chicken myocytes.

### Example 2

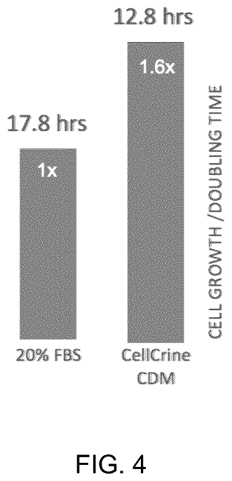

The proteinase and FGF growth factors in a serum-free culture media helps cells grow and proliferate better than culture media with serum, specifically fetal bovine serum (FBS). In another embodiment, as seen in FIG. 4, a graph of doubling time comparing muscle cells were grown in cell culture media with fetal bovine serum (FBS) at a concentration of 20% by volume and in serum-free culture media with a proteinase in the media at a concentration of 5 ng/ml and a concentration of growth factor FGF-2 at 1 ng/ml. The data for cell growth were normalized to one. The doubling time for muscle cells in the 20% FBS culture media was 17.8 hours at 1×. The doubling time for the muscle cells in serum-free media with the proteinase and FGF2 was 12.2 hours at a 1.6×. The muscle cells in serum-free media with the proteinase and FGF2 had significantly faster doubling times with denser cell spacing.

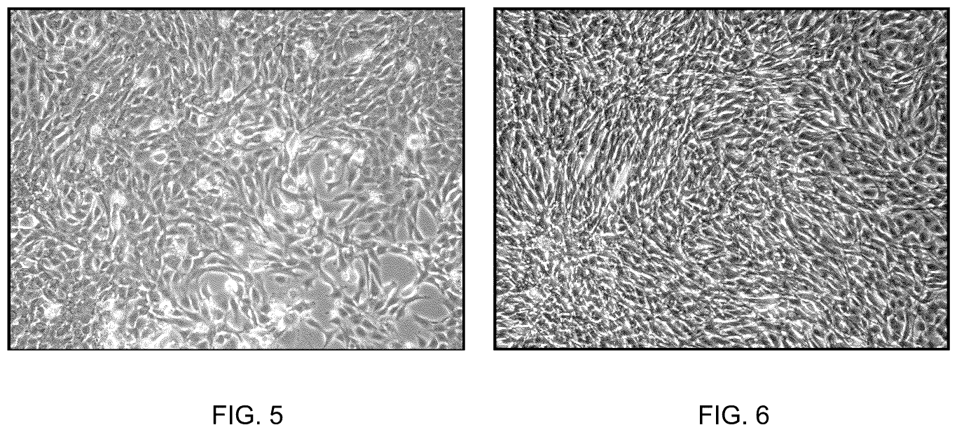

The muscle cells grown in 20% FBS are shown in a micrograph as FIG. 5 and the cells grown in serum-free media with the proteinase and FGF-2 media are shown in a micrograph as FIG. 6. As seen by comparing the micrographs, the cells grown in the in serum-free media with the proteinase and growth factor FGF-2 media are closer together and have more cells. The media with the proteinase and growth factor FGF-2 grows more cells in the same space in less time.

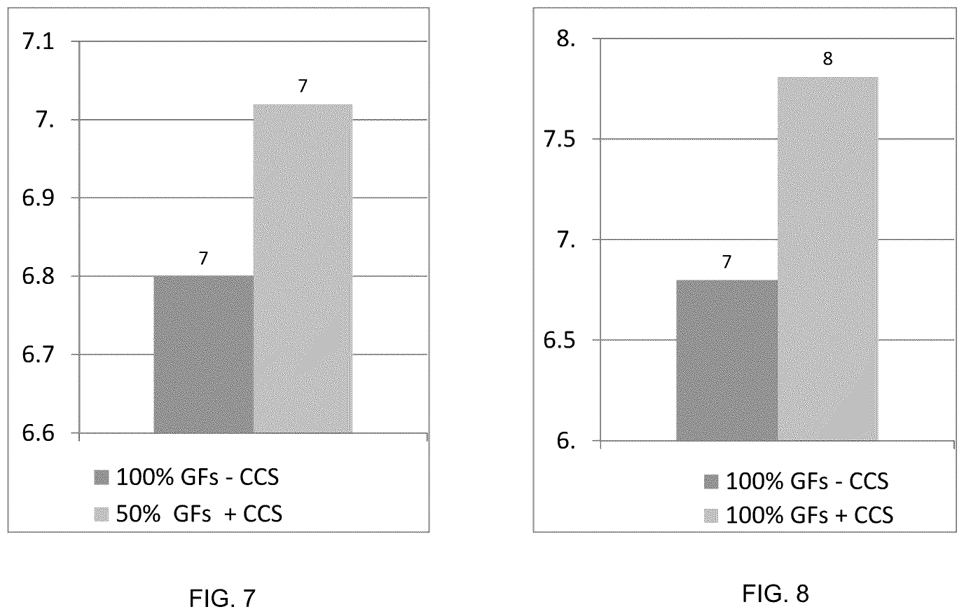

Cells grown in media with growth factors and with a proteinase grow better than cells grown in media with growth factors and without a proteinase. FIG. 7 shows that cells grown in media with a proteinase and growth factors grows better than cells grown in a media with twice the amount of growth factors and no proteinase, but otherwise identical. The cells grown in media with growth factors and without a proteinase resulted in 6.8 million cells per mL, while the cells grown with growth factors and a proteinase resulted in 7.1 million cells per mL. FIG. 8 shows that cells grown in media with growth factors and a proteinase grow better than cells grown in media with growth factors and no proteinase. The cells grown in media with growth factors and without a proteinase resulted in 6.8 million cells per mL, while the cells grown with growth factors and a proteinase resulted in 7.8 million cells per mL.

Other cell types have also shown increased growth and decreased doubling times, meaning that more cells are grown in the same space in less time. These cells include Porcine skin fibroblasts, which had a doubling time of 20.4 hours; Porcine skin keratinocytes which had a doubling time of 16.2 hours; primary Chicken myoblasts which had a doubling time of 24.9 hours; and mammalian myocyte cell line which had a doubling time of 12.8 hours. Each cell type has a different doubling time. This indicates that the cell type, i.e. myocyte, fibroblast, keratinocyte has unique interactions with the proteinase and the growth factors. The amounts of each of these compounds can be altered to optimize the results for culturing and propagating each type of cell. In addition to the cell types, the organism from which the cell is derived is affected by the proteinase and the growth factors. The cells from each organisms respond differently to the proteinase and the growth factors. Optimization for culturing, growing, and propagating cells can include utilizing different sources for the proteinase and growth factors.

All patents and published patent applications referred to herein are incorporated herein by reference. The invention has been described with reference to various specific and preferred embodiments and techniques. Nevertheless, it is understood that many variations and modifications may be made while remaining within the spirit and scope of the invention.

### REFERENCES

S. Löffek, O. Schilling, C-W. Franzke. Biological role of matrix metalloproteinases: a critical balance. European Respiratory Journal 2011 38: 191-208.
Page-McCaw A, Ewald A J, Werb Z. Matrix metalloproteinases and the regulation of tissue remodeling. Nat Rev Mol Cell Biol 2007; 8: 221-233.
Ra H J, Parks W C. Control of matrix metalloproteinase catalytic activity. Matrix Biol 2007; 26: 587-596.
Stricker T P, Dumin J A, Dickeson S K, et al. Structural analysis of the a2 integrin I domain/procollagenase-1 (matrix metalloproteinase-1) interaction. J Biol Chem 2001; 276: 29375-29381.
Saarialho-Kere U K, Kovacs S O, Pentland A P, et al. Cell-matrix interactions modulate interstitial collagenase expression by human keratinocytes actively involved in wound healing. J Clin Invest 1993; 92: 2858-2866.
Pilcher B K, Dumin J A, Sudbeck B D, et al. The activity of collagenase-1 is required for keratinocyte migration on a type I collagen matrix. J Cell Biol 1997; 137: 1445-1457.
Van Wart H E, Birkedal-Hansen H. The cysteine switch: a principle of regulation of metalloproteinase activity with potential applicability to the entire matrix metalloproteinase gene family. Proc Natl Acad Sci USA 1990; 87: 5578-5582.
Benbow U, Brinckerhoff C E. The AP-1 site and MMP gene regulation: what is all the fuss about?Matrix Biol 1997; 15: 519-526.
Vincenti M P, Brinckerhoff C E. Signal transduction and cell-type specific regulation of matrix metalloproteinase gene expression: can MMPs be good for you?J Cell Physiol 2007; 213: 355-364.
Egeblad M, Werb Z. New functions for the matrix metalloproteinases in cancer progression. Nat Rev Cancer 2002; 2: 161-174.
Bergers G, Brekken R, McMahon G, et al. Matrix metalloproteinase-9 triggers the angiogenic switch during carcinogenesis. Nat Cell Biol 2000; 2: 737-744.
Dean R A, Overall C M. Proteomics discovery of metalloproteinase substrates in the cellular context by iTRAQTM labeling reveals a diverse MMP-2 substrate degradome. Mol Cell Proteomics 2007; 6: 611-623.
Kleifeld O, Doucet A, Auf dem Keller U, et al. Isotopic labeling of terminal amines in complex samples identifies protein N-termini and protease cleavage products. Nat Bio-technol 2010; 28: 281-288.
Dean R A, Butler G S, Hamma-Kourbali Y, et al. Identification of candidate angiogenic inhibitors processed by matrix metalloproteinase 2 (MMP-2) in cell-based proteomic screens: disruption of vascular endothelial growth factor (VEGF)/heparin affin regulatory peptide (pleiotrophin) and VEGF/connective tissue growth factor angiogenic inhibitory complexes by MMP-2 proteolysis. Mol Cell Biol 2007; 27: 8454-8465.
McQuibban G A, Gong J H, Tam E M, et al. Inflammation dampened by gelatinase A cleavage of monocyte chemoattractant protein-3. Science 2000; 289: 1202-1206.
Rodríguez D, Morrison C J, Overall C M. Matrix metalloproteinases: what do they not do?New substrates and biological roles identified by murine models and proteomics. Biochim Biophys Acta 2010; 1803: 39-54.
Gutierrez-Fernandez A, Inada M, Balbin M, et al. Increased inflammation delays wound healing in mice deficient in collagenase-2 (MMP-8). FASEB J 2007; 21: 2580-2591.
Harvey, et al. Genes and Development 5:2375-2385, 1991; Pereira-Smith, J. Cell Physiol 144:546-9, 1990; Smith et al. Science 273:63-67, 1996.

1. A composition comprising:
a cell culture medium;
a proteinase; and
a growth promoting factor
wherein the doubling time of cells in the medium is the same or lower as compared to the doubling time of the cells in an otherwise identical composition which lacks the proteinase and has a higher concentration of the growth promoting factor.

2. The composition of claim 1, wherein the higher concentration of the growth promoting factor is about 2-fold to about 120-fold higher.

3. The invention of claim 2, wherein the proteinase is MMP-1.

4. (canceled)

5. (canceled)

6. The composition of claim 1, wherein the proteinase is heterologous to the cells.

7. The composition of claim 1, wherein the growth promoting factor is FGF-2.

8. (canceled)

9. (canceled)

10. The composition of claim 1, wherein the growth promoting factor is selected from FGF, TGFb, PDGF, VGF, IGF, and KGF.

11. (canceled)

12. The composition of claim 1, wherein the cells comprise myocytes.

13.-17. (canceled)

18. A method of culturing cells, the method comprising propagating cells in the composition of claim 1.

19. The method of claim 18, wherein the higher concentration of the growth promoting factor is about 1.25-fold to about 120-fold higher.

20. (canceled)

21. (canceled)

22. (canceled)

23. The method according to claim 18, wherein the proteinase is heterologous to the cells.

24. The method according to claim 18, wherein the growth factor is FGF-2.

25. (canceled)

26. (canceled)

27. The method according to claim 18, wherein the cells comprise myocytes.

28. (canceled)

29. (canceled)

30. (canceled)

31. (canceled)

32. (canceled)

33. The method according claim 18, wherein the cell culture medium comprises water, salt, amino acids, vitamins, glucose, heregulin, selenium, transferrin, insulin, ECM proteins, and ascorbic acid.

34. The method according to claim 18, wherein the doubling time of the cells is reduced as compared to the doubling time of the cells in an otherwise identical cell culture medium lacking the proteinase.

35. The method according to claim 18, wherein the doubling time of the cells is reduced as compared to the doubling time of the cells in an otherwise identical cell culture medium which lacks the proteinase; and comprises an about 20- to about 120-fold decrease in the concentration of the growth factors.

36. The method according to claim 18, the method further comprising optimizing the concentration of the proteinase to achieve the lowest doubling time for the cells.

37. The method according to claim 18, the method further comprising optimizing the concentration of the growth factor to the lowest concentration that supports the lowest doubling time for the cells.

38. The method according to claim 18, the method comprising culturing the cells in the cell culture medium for at least 1 day.

39. The method according to claim 18, the method comprising culturing the cells in the cell culture medium for at least 2 days.

40. The method according to claim 18, the method comprising culturing the cells in the cell culture medium for at least 5 days or longer.
